(19) United States
(12) Patent Application Publication
(10) Pub. No.: US 2015/0216921 A1
(43) Pub. Date: Aug. 6, 2015

(54) BASE POWDER FROM THE RED RASPBERRY RUBUS IDAEUS AND ACTIVATED MICRONIZED ZEOLITE FOR ATTENUATING NICOTINE ADDICTION, METHOD FOR THE PREPARATION THEREOF AND USE THEREOF

(71) Applicant: University fo Hidalgo, Pachuca (MX)

(72) Inventors: Javier Isaias Alanis, Morelos (MX); Ricardo Perez-Pasten Borja, Pachuca (MX); Santiago Filardo Kerstrupp, Pachuca (MX); Alfonso Atitlan, Pachuca (MX)

(73) Assignee: University of Hidalgo, Pachuca (MX)

(21) Appl. No.: 14/351,846

(22) PCT Filed: Oct. 12, 2011
(86) PCT No.: PCT/MX2011/000124

§ 371 Date: Jan. 6, 2015

(51) Int. Cl. A61K 36/73; A61K 9/16; A61K 33/06; A23L 1/304; A23L 2/52

(52) U.S. Cl. CPC A61K 36/73; A23L 1/3045; A23L 2/52; A61K 33/06; A61K 9/1682; A23V 2002/00; A61K 2236/37; A61K 2236/51

(57) ABSTRACT

The present invention relates to a base powder for preparing functional beverages, soft drinks or an additive for food products, which is made with red raspberry from Rubus idaeus and with clinoptilolite zeolite contained in the ZAM® (activated micronized zeolite). The powder is prepared with dehydrated fruit juice. The raspberry juice extract is obtained by slowly heating (without exceeding 70° C.) a pot containing the ground fruit or a receptacle with fruit, using the bain-marie method, without exceeding 70° C. The juice is stabilized, dehydrated, optionally mixed with sugar substitutes, sweeteners or fiber, and, lastly, homogenized. The object of the present invention is to provide a food-grade base powder of natural origin that is totally different from current presentations of red raspberry on the market. Likewise, the present invention seeks to utilize the therapeutic properties of said fruit as a coadjuvant in controlling active and passive tobacco consumption, nicotine detoxification and nicotine addiction, and also detoxification in the case of other toxic substances in tobacco smoke, as an antioxidant, antimutagenic, anticancer agent and immunostimulant. It is 100% rehydrateable in water, easy to transport and has a shelf life in excess of one year.

### FIELD OF INVENTION

The objective of the present invention is to protect the intellectual property, ensure the product and the process to elaborate the base powder to prepare functional beverages, refreshing beverages or as additive for food products and in encapsulated products or tablet form, made with fruit of raspberry (Ideaus Rubus) and ZAM® (activated and micronized zeolite), with capability to attenuate the addiction and the withdraw syndrome to the nicotine.

### BACKGROUND OF THE INVENTION

The use of plants, fruits and minerals favor health and contribute in the fight against diverse illness, therefore an increasing interest exists from consumers; mean while the industry offers continuously new food products that respond to the population demand. The abundant content of the ellagic acid that the rubus idaeus has make it as an ideal fruit to propose it as base for an antioxidant product, antimutagenic product, anticancer product(in some types of cancer), and as an alternative to the tobacco addiction problem; its efficacy as antioxidant and antimutagenic, as well as the modification of the nicotine metabolism and the addictive effects of it shows the need to create other means to obtain the benefit effects of this fruit, since when it is consumed in its natural form could cause repulsion in daily use of the fruit not ripe. In other side, the ZAM® zeolite-clinoptilolita activated and micronized of high quality and purity, act as a functional vehicle (carrier) for fruit extracts, herbal extracts, vitamins and organic and synthetic molecules, used in pharmaceuticals preparations, food supplements and cosmetics. So its application as a vehicle of food supplement puts the ZAM® (activated and micronized Zeolite) as a mechanism that allows to carry ellagic acid, in the raspberry in a major quantity and mayor quality, potentiating the effects as antioxidant, antimutagenic and anti-carcinogenic; in addition, the ZAM® (activated and micronized zeolite) also has antioxidant properties, anti-mutagenic and anti-carcinogenic, as well as the detoxify capacity catching nicotine and some of the other toxic substances in the tobacco smoke.

The benefic effect of the fruits and minerals, as well as the growing interest of the consumers for their health condition, promotes that the industry offer continuously new food products to respond to the demands of the population. The concept of functional foods, marks the start of the development of food products to improve the health and reduce the risk of becoming sick. A functional food is that one that has components that provides benefits for health beyond of a basic nutrition, it is about a food product or beverage that produce physiologic benefits, in function of the content of one or several natural active ingredients, aggregated or modified, changed by technology or biotechnology.

The need to have food products that are more benefic for health, also is supported by the socioeconomic changes and demographics, that are happening in the population, reason why researchers, professionals in health and food industry are looking forms to control these changes in a more efficient way. An example of this is the use of the phytochemicals, so called too as phytodrugs that have taken boom in the worldwide therapeutic arsenal.

Nowadays, the nutrition is experiencing a fast chance in certain interest areas. The nutritional deficiencies aren't anymore the main priorities of the research and nowadays the interest is in the relation between the feeding and the non transmissible chronic diseases and the effects of the nutrition over the cognitive, immune functions, the work ability and athletic performance. In the other side, the consumers are increasingly aware of self care and are searching in the market those products that contribute to health and wellbeing. These foods, that promote health, generically have been named functional foods and the enterprises that produce them show a fast worldwide expansion.

The functional foods have the particular characteristic that some of their components affect organism functions in a specific and positively way, promoting a physiologic or psychological effect beyond of their nutritive traditional value. Their additional effect can be their contribution to keep the health and wellbeing or the diminish in the risk of getting sick.

Another example is the use of the phytochemicals, that have taken boom in the worldwide therapeutic arsenal. The phytopharmacology and the phytotherapy, are recognized and used by the traditional medicine. In the plants or in their parts, as well as in the fruits, that are used to generate the functional foods, the active ingredients are always in biologic equilibrium because of the presence of complementary substances, that are potentiated between each other, that they are not accumulated in the organism; and fundamentally because their not desired effects are limited, and also they employ low investment technologies and raw materials with the consequence diminish in costs. (Hernandez, 2003).

In particular, alcohol and tobacco are the most abused commonly used substances. The extract of Hypericum perforatum L, is an example of pytotherapy compound, has being used for the treatment of low to moderated depression, caused by the abuse of the substances, the studies were focused on the alcohol and nicotine dependence (Tayfun 2001).

The smoke of the tobacco is a complex mix of chemicals agents which besides of being toxic, mutagenic and carcinogenic, contains nicotine, which is the responsible of the smoking addiction, is the component that the smokers are essentially looking for with the use of tobacco. After the nicotine enters in the circulatory system, the active nicotine is eliminated from the organism as cotinine form. The responsible enzymes for the catalysis in this reaction are in the cytochrome P450 2A6 (in humans) and, together with an aldehyde oxidase, conform the limiting stage of the nicotine metabolism. The persons that shows deficiencies in the function of this reaction, because of the presence of inactive alleles of the enzyme CYP2A6, shows a diminished capacity to metabolize the nicotine; it has being observed that these persons are less susceptible to smoke. Pilot studies have demonstrated that the inhibition of the activity of this enzyme can reduce the smoking frequency. Additionally to the nicotine, the CYP2A6 in humans, metabolize other xenobiotic compounds, such as tobacco nitrosamines and other toxic components. Recent experimental evidences show that the inhibition of the CYP2A5 enzyme, similar to CYP2A6 in human, reduces dramatically the tumoragenesis provoked by the nitrosamine in mice.

Until now, the studies that suggest the influence of the nicotine metabolism, and its relationship with the development and maintenance of the addiction to it are extense; meanwhile there are few experimental evidences that relate the changes in the nicotine metabolism and its effect in addictive conduct; and the studies that analyze nicotine metabolic inhibition, are mainly focused on the toxic effects of this alkaloid. According to Denton et. al. (2004) the analog β—(nicotirine), product of the nicotine combustion, inhibits the CYP2A6 enzyme, in the same way that the quinidine that, at high dosages, is able to inhibit this enzyme (Hirano and Mizutani, 2003). Rahnasto et. al. (2005) developed a β—(naftalene) analog series, all capable to inhibit specifically the CYP2A6. No one of the mentioned cases, have related this property with the addictive conduct. In other side, there are works in which they have demonstrated that some polyphenolic agents are capable to protect from mutagenesis and carcinogenesis induced by nicotine, thanks to their capacity to inhibit their metabolism. Miller et. al. (1993), demonstrated how the polyphenolic compounds of the natfoflavones family, can reduce the mutagenesis inducted by nitrosamines in s.tyhpimurim, this effect is explained by the inhibition of the set of the enzymes of the citochrome P450. Castonguay et. al. (1993) proved that the polyphenolic compounds coming from of the diet, are able to reduce the tumorogenesis in mice that were exposed to nitrosamines; meanwhile Zhang (1993) have related the protective effect with the enzyme CYP2A6 inhibition. In this sense, the ellagic acid (EA) (2,3,7, 8—tetrahidrobenzopirano [5, 4, 3—cde] benzopiran-5-10 diona) is a polyphenolic compound that is located in fruits like raspberry, cranberry, pomegranate, similar fruits, in the walnut and in wood; and has been demonstrated that possesses different pharmacologic effects like anticarcinogenic, antibacterial and anti-inflammatory. In in-vitro studies it has being observed that affects the expression of genes p53 y 021 and produces detention in phase G1 and apoptosis in cancer cells. Also, inhibits the growth of cancer cells modifying the activity of the enzymes topoisomerase I and II, girase and polymerase. Also can act as antioxidant against large chain peroxil radicals, and whit it prevent the propagation of the mediated reactions of free radicals.

The EA intra peritoneal injection of is not effective against tumorogenesis in lung of benzo [a] pyrene. In contrast, it have been observed a inhibition of 90% in the lung tumorogenesis of benzo [a] pyrene in mice feed by EA at a dose of 400 mg/kg of EA. Boukharta (2002) demonstrated that the EA is capable to reduce the tumorigenicity of the nitrosamine NNK in a concentration dependent manner. At a dose of 4g/kg inhibited tumor multiplicity by 54%. Multiple observations have led in different hypotheses of the mechanisms preventing the tumorogenesis of EA, the main one is based in the capability to inhibit the activation of diverse carcinogens mediated by CYP2A6, the induction of enzymes of phase II involved in detoxification of the body, in addition it has been demonstrated that the EA is a modifier of the genotoxicity induced by the nicotine in lymphocytes T (Stoner, 2001; Lei 2001; Falsaperla, 2005; Adluri, 2007).

The EA, by its induction of various detoxification enzyme systems result as a potential inhibitor of nicotine addiction and syndrome of withdrawal, as well as in the detoxification of many toxic substances in tobacco smoke. On the other hand the EA is a molecule capable to precipitate proteins and alkaloids (Public Health and Nutrition, 2006); the nicotine is an alkaloid, which when it is precipitated it would be limited to get to their metabolic cycle, with consequent limitation of the addictive process.

In summary, ellagic acid, is an example of one type of polyphenolic compound that acts as a phytochemical; has a variety of biological activity, antioxidant, anti-inflammatory, and anti-fibrosis effect; induces diverse detoxification enzyme systems, and inhibits reactions by blocking nitrates. Also blocks various carcinogens, reducing the benzopyrenes bioavailability in the gastrointestinal tract. It also acts as a modifier of the nicotine-induced genotoxicity, finding a positive modulation of the damage it causes to T cells. In other studies it was found that the raspberries, in their natural state, can lead to significant increases in removing nicotine, favoring the process of smoking cessation (Milano, 2001).

Likewise, it has been identified that the zeolites, as volcanic minerals originally, have a powerful detoxifying action, being the only known minerals negatively charged on earth that, naturally, attract and adsorbed positively charged toxins.

By its composition, the zeolite—clinoptilolite is a structured silico—aluminate of tetrahedral crystals, often stacked in pairs double or quadruple. The Zeolite-clinoptilolite has a rich network of micropores and microchannels filled with semi-exchangeable cations of calcium, sodium, magnesium and potassium, which may be exchanged with free radicals nitrogen and oxygen.

Different research studies support the anti-cancer, antioxidant and detoxifying action of the zeolite. Kresimir (2000) showed the zeolite-clinoptilolite as an adjunct in anticancer therapy and in 2002 (Kresimir, 2002) demonstrated the immunostimulatory effect of natural cliniptilolita as a possible mechanism of its antimetastatic activity; Kresimir et.al. (2004) demonstrated an antioxidant and inmunostimulating effect of the natural zeolite, while Grace and Kresimir (2004) demonstrated the antiviral properties to the clinoptilolite. Tomasevic (2005) demonstrated the medical application for extracting lead with natural zeolite-clinoptilolite.

To reduce the toxicity of tobacco, several combustion systems have being used (Baker, 2006 and Ding et al. 2007). In these systems, a catalyst has been added, which is mixed with the tobacco or it is used as filter in the tip cigarettes. Most of these systems involve different materials of zeolite or alumino-silicates. In the first attempts (Seeofer et al., 1980), a compound was used with the formula M2M′Ru06, where M was a divalent metal, M′ is a trivalent rare earth metal, Ru having a valence of 5, and M and M′ are capable of forming a network with Ru ions; this compound, when mixed with tobacco or is incorporated in the cigarette paper or in the filter, helps reduce the NO and CO of the tobacco smoke. Rongved (1997) described a compound to produce less toxic substances as carbon monoxide in the flue gas, by adding inert, inert and solid catalysts, stable and non-contaminating in a mixture with the tobacco or as a filter, using a metal catalyst, as vanadium pentoxide, molybdenum trioxide or rhodium oxide. More recently, Li et al. (2003), describe the use of nanoparticles of Fe2O3, CuO, TlO2, CeO2, Ce2O3, Al2O3, Y2O3 doped with Zr, Mn2O3 combined with Pb, to increase the conversion of CO to CO2. On the other hand, it has been described that the potassium organic salts can be used as additives to tobacco and, in the burning cigarette, may reduce the yields of CO and nicotine, (Li et al., 2003). In 2004, Li et al, describe the use of an oxidizing catalyst, based on iron oxide nanoparticles, which is generated in situ when the cigarette is burning, and is able to increase the conversion of CO and NO to CO2 and N2, respectively.

The use of zeolites and other additives aluminum-silicates has been described by several authors. Cvetkovic et al., (2002) described the use of a catalyst based on a combination of Cu-zeolite and ZSM-5 to reduce the amount of NO and NOx in the conventional tobacco smoke. This catalyst can be added to the cigarette filter or can be directly mixed with tobacco. This proposal is based on the adsorption or diffusivity properties of the material. Meier et al., (2001 and 1999) also describe the use of zeolitic materials as additives, and Xu and Zhu (1985); its about mesoporous materials (MCM-48, NaA Zeolite NaY KA and NaZSM-5, SBA-15 y MCM-48) used as additives to tobacco to cigarettes, and can reduce the content of nitrosamines, by selective adsorption, into the smoke snuff sucked by active smokers directly from the cigarette. Gao et al., (2009) also describe the use of CAS-1 in the cigarette filter for selectively absorbing nitrosamines. Yong et al., (2006) proposed the use of MCM-48 and Ce-MCM48 to reduce the content of polycyclic aromatic compounds in smoke tobacco main stream. Chen et al., (2006) presented NaY zeolite carbon, which activates and oxidized carbon nanotubes in the filter cigarette and was found an important reduction of tar and nicotine. Also reported by (Branton et al., 2009) the important reductions in fumes composition of activated carbon when the zeolitic compounds are introduced into the filter tip.

Other studies have reported the effect of three commercial zeolites (Husy, Hb, HZSM -5) and a synthesis of catalysts, Al—Si—MCM41 (crystalline mobile material, silicate obtained by a template mechanism) in the composition of the mainstream smoke obtained from one brand of cigarettes of Virginia tobacco has been studied. Catalysts were seen directly mixed with tobacco. The results suggest that the inclusion of Catalyst Al- Si-MCM41 directly with the tobacco in cigarettes could be an efficacy procedure for reducing the presence of many toxic and carcinogenic compounds in the mainstream smoke.

Therefore, the ZAM® (activated and micronized zeolite potentiates the effects of ellagic acid and in general of the raspberry powder, acting as a vehicle increasing the bioactivity and bioavailability of raspberry powder and its components. The patent applicants have shown that the capability of pure ellagic acid and reconstituted juice based powder of raspberry and also the same powder used as an additive in other food products, the first motive of the present patent, are capable to module the addictive conduct and the nicotine withdraw syndrome in dosage-dependant mice. In the other side, the patent applicants, also have demonstrated that ZAM® (activated and micronized zeolite), manufactured by Granding International SA de CV, from natural zeolite-clinoptilolite, has an average particle size of 3 microns, a surface area of 24 m2/g, and has been thermally activated, so its bioactivity and bioavailability are increased, allowing it to act as a vehicle (carrier) functional for raspberry powder, also, besides being a potent antioxidant, anticarcinogenic and being able to trap nicotine and other toxic substances from tobacco smoke, because is an absorbent of toxins such as NH3, hydrocarbons, Cl-C4, CO2, H2S, SO2, NOx, aldehydes, besides heavy metals like lead, cadmium, cesium, mercury, etc. is the second motive of applying this patent.

To verify product novelty characteristic, a search was performed on different databases to determine the state of the art for the product under this application. The databases consulted were: National Bank of Patent (Banapanet), United States Patent and Trademark Office (USPTO) and the European Patent and Trademark Office (EPO)in the combination of words, technical terms or synonyms and/or codes of classification, also as: POWDER AND RED RASPBERRY AND FUNCTIONAL DRINK, ZEOLITE and CLINOPTILOLITE and MICRONIZED. From this search more than 200 documents were obtained, but only those were selected those that seemed to be related to the protected subject matter of this document, and are listed below:

a. Patent Application: KR20040010390, Date: 31-01-2004;

Anti-influenza Virus agent and composition containing the same and food or drink; Kazafumi, S. et al.

In this application a standardized formula is obtained from the following fruits and plants: Rubus idaeus, Fragara vesca, Rubus fruticosus, Ficus cariaca, Chenopodium album, Agrimonia eupatoria, Eucalyptus globulus, Prunus persica, Malus pumila, Viola odorata, Lindera umbellata, Paullinia cupana, Buddelia officinalis, Commelina communis, Capsella bursapastoris, Rabdosia japonica. Which is obtained by dehydration juice and hydroalcoholic extracts at temperatures of 4 to 5° C., prioritizing stability of thermolabile phytochemical agents. The biological activity attributed to it is that of an anti-influenza (antiflu) agent, attributed to immunostimulatory properties.

b. Patent Application U.S. 2004/0013749 A1, Date: 22-01-2004;

Antioxidant and immune boosting composition and methods of using; Young, D. G. et al.

In this application a standardized formula raspberry (Rubus idaeus) is obtained, which is obtained by dehydration of the juice temperature of 4 to 5° C. and pH below 5, prioritizing stability thermolabile phytochemical agents. The biological activity attributed to the agent is that of an antioxidant and immunostimulant agent. The standardization of the formula is made based on the antioxidant capacity of said extract.

c. Patent Application: PA/a/2005/005479; PCT/EP2003/012975;

Date: 23-01-2005, Nutraceutical compositions which comprise epigallocatechin gallate and raspberry ketone; Weber, P. et al.

This application a standardized formula gallate epigalato and its ketone obtained from raspberry (Rubus idaeus). Which is obtained by dehydration of the hydroalcoholic extract fractionated at temperatures of 4 to 5° C. and a pH below 5 and extraction methods, prioritizing stability thermolabile phytochemical agents. The biological activity attributed is that as preventive against cancer and neoplasias. The standardization of the formula is made based on the concentration of gallate-epigalato.

d. Patent: PA/a/1997/002984; 04/24/1997 Date: Removal of iron and manganese by adsorption-oxidation on natural zeolite, method of forming the contact medium and regenerate its adsorption capacity.

In this patent a technique and methodology is registered in which the physical and chemical characteristics of the clinoptilolite type zeolite are used to eliminate or removal of iron and manganese metal ions that are found in the water of some underground sources of supply.

The product for which a this patent is applied has different obtaining methods as the aforementioned documents, and also different applications are described; is a base powder of raspberry with zeolite to produce a functional beverage, as an additive to other foods or other products, that is obtained from a standard fruit juice extracted from the fruit by the method of pot or maría bath at temperatures above 50 to 70° C. This juice is then dehydrated by a spray dryer method at a temperature of 110° C. This procedure favors the thermo-stable components especially ellagic acid. Also ellagic acid is the component to which it is attributed the biological activity, which aids in the treatment of tobacco addiction, the body immunostimulation, antioxidant, antitumor and anticancer activity, which is enhanced by adding the ZAM® (activated and micronized zeolite), by its activity as a carrier, as a detoxifying, immunostimulant, antioxidant and anticancer.

Therefore the above documents do not represent duplicity, consequence or immediate deduction, or variations of patent applications recovered from the homologous search patents, and consequently the product for which registration patent is requested, meets innovation condition. Since in the previous documents was found that in obtaining raspberry products an extremely low temperatures were used and drying at low temperature, and no heating or drying at high temperatures, as it is being in the obtaining process of the product object by this patent; further that none of the cited cases, privileges the presence of ellagic acid nor the biological effect attributable to it, as an aid in the detoxification of nicotine and, therefore in the treatment of anxiety during the quitting smoking process and in the treatment of addiction to nicotine; even more no report of raspberry in combination with clinoptilolite zeolite, much less the carrier activity of ZAM® (activated and micronized zeolite) for the components of raspberry powder; none mention the improved bioavailability and participation as detoxifying of the nicotine and other toxic substances in tobacco smoke.

### DETAILED DESCRIPTION OF THE INVENTION

The Product object of the present invention, is obtained from the combination of the ZAM® (activated and micronized zeolite) and raspberry powder (according to the process registered in the Mexican Institute of Industrial Property, in patent application number Mx/a/2010/000827 dated 21/ENE/2010, folioMX/E/2010/004489). Therefore some modifications are specified that improve the protected product object of the mentioned application. The base powder works to prepare functional beverages, refreshing drinks, or as an additive for food products and other products for tobacco addiction control. The base powder is made from pulp and dried strawberry raspberry juice fruit and ZAM® (micronized and activated zeolite) that serves as a vehicle (carrier) to improve the bioavailability of ellagic acid and other ellagitannins in the raspberry, as well as detoxifying agent for nicotine and other toxic substances present in tobacco smoke.

The fruit is selected by assessing the clear red color, making sure that it is free of any toxic substances in accordance with the allowed tolerance by the Secretariat of Health and Welfare for processing fruits.

The raspberry juice extract is obtained using the method of kettle or water bath method, or some other alternative method.

Once raspberry juice extract is obtained, the powder is obtained by a drying process, for example by aspersion usinn and equipmento of Spray-dryer type, or a lyophilization process, or any other alternative method.

Optionally, the powder obtained form raspberry juice extract can be added with any allowed sweetener, for example sugar or aspartame.

To the powder obtained from extract raspberry juice, added with any allowed sweetener, for example sugar or aspartame, an anti-humectant is added, such as silicon dioxide or tricalcium phosphate. The ingredients are mixed and homogenized.

Once adding any allowed sweetener and whit an allowed antihumectan, the powder obtained from the juice extract of raspberry ZAM® (activated and micronized zeolite) is added. The ingredients are mixed and homogenized to obtain the final base powder.

Optionally, the homogenized product may be packaged in glass jar with twist off cap and plastisol seal, or in polyethylene-aluminum laminated pouches and glassed paper, or any other allowed packaging method.

Additionally, the powder reconstituted with water, preserve its minerals content such as calcium, iron, aluminum, magnesium, potassium, silicon, sodium, manganese, sulfates and phosphates, carbohydrates and nitrogenous compounds; as well as vitamins A, B1, B2 and C and chemical components of the ZAM® (activated and micronized zeolite), SiO2, CaO, K2O, MgO, Na2O.

Also, the powder product has a shelf life superior than one year, keeping its original properties.

The base powder is rehydrated with water instantly and can be used to prepare functional beverages, refreshing drinks or as an additive in food preparations or other products, retains all therapeutic properties, such as we can mention: auxiliary in tobacco addition control, based in the content of ellagitannins, specifically the ellagic acid and the ZAM® (activated and micronized zeolite); promote the precipitation and elimination of nicotine, helps to maintain low levels of anxiety caused by withdrawal syndrome in the suspending use of tobacco, and with this the administration of nicotine, as well as helps to diminish the nicotine addiction, these make it in an excellent support to quit tobacco smoking, and because the antioxidant, antimutagenic, anticancer, and inmunostimulatin properties, helps to fight some of diseases caused by tobacco smoking and helps to keep them free of smoking.

The base powder is instantly rehydrated with water and can be used to prepare functional beverages, refreshing drinks or as an additive in food preparations or other products, retains all its minerals like calcium, iron, aluminum, magnesium, potassium, silica, sodium, manganese, sulfates and phosphates, carbohydrates and nitrogenous composite, plus vitamins A, B1, B2 and C, as well as the chemical components of the ZAM® (activated and micronized zeolite), S1O2, CaO, K2O, MgO, Na2O.

The base powder is instantly rehydrated with water and can be used to prepare functional beverages, refreshing drinks or as an additive in food preparations or other products, and retains its bioactivity and increased bioavailability, activity provided by the ZAM® (activated and micronized zeolite), giving it the quality of being an excellent antioxidant to fight free radicals.

The base powder is instantly rehydrated with water and can be used to prepare functional beverages, refreshing drinks or as an additive in food preparations or other products, and provides a bittersweet sui generis solution.

The details and characteristics of the process to develop this base powder from raspberry and ZAM® (activated and micronized zeolite), to obtain a functional beverage, refreshing drinks or as an additive in food preparations and other products, are shown in detail in the following description:

Process to obtain of base powder to prepare functional beverages, refreshing drinks or as an additive in food preparations or other products made with red raspberry, Rubus idaeus and ZAM® (activated and micronized zeolite).

Stage 1—Main raw material

Red raspberry fruits and ZAM® (micronized and activated zeolite)

Stage 2—Selection, cleaning and weighting the fruit.

Raspberry fruit is selected so that it is not too ripe, evaluating its clear red color. Bunches of stems are separated and remove the leaves, the peduncles are removed; these are usually divided into two or three pedicels, and the fruits are placed in a clean surface, as for example a clean tray or other similar. The Raspberry bunches are weighed before dehydrating.

Stage 3—Juice extract obtaining and standardization and powder juice extract production.

Raspberry juice extract is obtained by using any of the known methods for example:

1) The method of Marmite—. The fruit previously crushed is placed in a marmite, which is slowly heated but not exceeding 70° C., in such way that the fruit juice begins to flow, during one hour for every 3 kg of fruit approximately. This method prevents that the fruit experience excessive overcooking, that destroys both the color and the fresh taste, and favour a mayor concentration of ellagic acid in the powder. Once the fruit has released plenty of juice, it is crushed again. Once the fruit juice is extracted it is filtered by passing through medium pore filter paper.

2) The water bath method. —The fresh fruit is crushed with a mortar or other means in a large bowl. The fruit bowl is placed in a water bath without exceeding a temperature of 70° C. to 80° C., until the fruit juice begins to flow. Optionally, once extracted fruit juice is obtained it could be filtered using filter paper medium pore.

Once the juice extracted, to obtain the powder, the extract is subjected to a drying process, for example by aspersion using a Spray-dryer equipment type, with an inlet temperature of 110° C. and outlet of 80° C., with an air flow of 600 ml/min and 30 millibars vacuum. It can be done also by lyophilization or any other known method.

Stage 4—. Using anti-humectants.

To the formulation obtained in Stage 3, is added with any allowed antihumectant, for example, silicon dioxide or tricalcium phosphate. Additionally any allowed sweetener may be added, for example, sugar or aspartame.

Stage 5—. Use of ZAM® (activated and micronized zeolite and homogenization in the formulation of the base powder.

To the formulation obtained in Stage 4, ZAM® (activated and micronized zeolite) is added For 250 g of powder raspberry 20 g of zeolite ZAM® are added. This is mixed and homogenized to obtain the final product.

Stage 6—Packaging base powder formulation.

Optionally, the homogenized product may be packaged in glass jar with twist-off cap and plastisol seal, or in polyethylene-aluminum laminated pouches and glassed paper, or by any other allowed method. This way the product retains its properties for more than 1 year, as the study of shelf life of the product was subject to, in extreme temperature and humidity conditions, correctly packed and in jars and it maintain its original properties.

### PRACTICAL EXAMPLE FOR CARRYING OUT THE INVENTION

The present example is illustrative and not limiting, such as one skilled in the art, will understand there are variants that fall within the protection of the present invention.

Process to obtain a base powder to prepare functional beverages, refreshing drinks or to be used as an additive for food preparations or other products, made with red raspberry, Rubus idaeus and ZAM® (activated and micronized zeolite).
1) Raspberry fruit with adequate ripeness condition and sanitation is selected.
2) Fruit leaves and stems are removed.

3) Raspberry juice is extracted by placing fresh fruit previously crushed in a kettle. The kettle is heated slowly without exceeding 70° C. until it starts flowing fruit juice, during approximately 20 minutes per kilogram of fruit. This method for extracting juice, avoid fruit experiment excess overcooking, and any destruction of both the color and the fresh taste and the medicinal properties and at the same time allows a higher concentration of ellagic acid. When the fruit has released plenty of juice, it is crushed again.
3) Optionally, the fruit juice is filtered by passing through medium pore filter paper.
4) After extraction of the juice, it is subjected to spray drying process using a spray-dryer equipment, with an inlet temperature of 110° C. and outlet temperature of 80° C., and an air flow of 600 ml/min and vacuum of 30 millibars.

6) To prepare the base powder, the spray dried raspberry juice powder is used, and can be prepared for example, in some of the following presentations, mixing the following proportions:
a) Base powder unsweetened light—. The product consists of 100% powder, obtained from raspberry juice extract using the spray drying.
b) Powder base with sweetener.—Were mixed in the following proportions, spray dried obtained extract raspberry juice 50% and 50% aspartame.

7) In each of the presentations described above, food grade anti-humectant is added to them, as silicon dioxide or tricalcium phosphate, among others.

8) The ingredients of any of the formulations described above and the anti-humectant are mixed with ZAM® (activated and micronized zeolite) and homogenized to obtain final base powder.

9) The homogenized final base powder, is packaged in glass jar with twist-off cap and plastisol seal or in polyethylene-aluminum pouch envelopes and glassed paper.

Main uses of base powder to prepare functional beverages, refreshing drinks or as an additive to food preparations or other products made with red raspberry Rubus idaeus and ZAM® (activated and micronized zeolite).

The powder product corresponds to a natural functional food, nutritional order, which can be used to prepare functional beverages, refreshing beverages as an additive in food preparations and in the preparation of other products.

1. The product is rehydrated with water instantly providing a sui generis solution bittersweet flavor.

2. The product may have various presentations, such as: a) base powder unsweetened light and b) base powder with sweetener or sweetener, among others.

3. In the case of the base powder presentation with sweetener or sweetener, sugar can be used or fructose or aspartame.

4. The base powder used to prepare functional beverages, refreshing drinks, as an additive in food preparations or other products, uses ZAM® (activated and micronized zeolite) as a vehicle (carrier) when it is ingested; also the ZAM® has the function of detoxifying, to carry, selectively nicotine and other toxic substances present in the tobacco smoke, to be eliminated.

5) The base powder can be used to prepare functional beverages, refreshing drinks, and also can be used as an additive in food preparations or other products. after reconstituted with water, fruit juices, any kind of milk, among other ingestible substances, except alcohol, preserves all the fruit properties: color and flavor, as well as the therapeutic properties of the raspberry together with the ZAM® (activated and micronized zeolite).

6. The base powder reconstituted with water retains its therapeutic properties, we can mention such as: antioxidant, anti-mutagenic, anti-carcinogenic and inmunostimulant, also in the detoxification for nicotine use and other toxic substances present in the tobacco smoke and in removing heavy metals in various forms presentations, activity based in the content of ellagic acid and zeolite-clinoptilolite; helps in the withdrawal syndrome control associated with anxiety provoked by the suppression of nicotine, and therefore allows controlling nicotine addiction. Therefore the intake of the power is recommended not only for people with nicotine addiction because of the cigarette consumption, but also for non-smokers people exposed to tobacco smoke, based in its function as an auxiliary in the control of anxiety and in the detoxification of nicotine and other toxic substances present in the tobacco smoke. The mentioned properties in raspberry powder, ellagic acid and ZAM® (activated and micronized zeolite) have been demonstrated by the patent applicants in different biological models; both nicotine withdrawal induced syndrome, and addictive behavior to it in animals, together with the anxiolytic effect. Likewise it has been demonstrated that the product is auxiliary in the withdrawal syndrome treatment, specifically helping with the anxiety, and also helps to control the addictive behavior in humans.

7. Using raspberry powder and pure ellagic acid (Sigma Aldrich) two experiments in mice were developed, one was to evaluate the effect of those in the anxiety caused by nicotine withdraw syndrome; and the other was to evaluate the nicotine addiction. Based on the process mentioned in the Mexican Institute of Industrial Property in patent application number Mx/a/2010/000827 record dated 21/ENE/2010, folio MX/E/2010/004489. In the first case the high-cross test was used and in the second test site selectivity paradigm. Nicotine was administered to mice for 14 consecutive days and when it was suspended, anxiety was developed. It was demonstrated that ellagic acid (EA) and raspberry powder (RP) significantly reduce signs related to anxiety caused by the refraining from exposure to nicotine, also raspberry powder reduces the natural anxiety that was generated in the group control mice, which means that an anxiolytic effect was obtained, even without withdrawal syndrome of nicotine. Furthermore, it was demonstrated that nicotine is able to condition the behavior of mice, in order to receive more stimulation from it; meanwhile the administration of EA and RP are able to modulate this behavior in a significant way.

8. It was demonstrated that the product is an auxiliary in the withdrawal syndrome control, particularly in relation to anxiety; and in controlling nicotine addiction in humans, using an experiment in which persons used different treatments with obtaining different results as follows:

9. Treatment 1:—Two persons, a man and a woman of 54 and 38 years respectively, used ZAM ® (activated and micronized zeolite) at doses of 5 g daily during 14 days.

10. Treatment 2:—Two persons, a man and a woman of 45 and 47 years respectively, used the raspberry powder recorded in January 2010 in the Mexican Institute of Industrial Property (patent application number record Mx/ a/2010/ 000827, at folio MX/E/2010/004489). At a dose of 5 g per day during 14 days.

11. Treatment 3: A person, a man of 63 years used a commercial product of raspberry, with similar characteristics to the raspberry powder, mentioned in the previous section, in combination with ZAM® (activated and micronized zeolite) at dose of 5 g per day.

12. Treatment 4: Two persons, a woman of 57 and a man aged 53, used raspberry powder registered in the patent application number Mx/a/2010/000827, folio MX/E/2010/004489) in combination with ZAM® (micronized and activated zeolite) in a dose of 7 g per day during 14 days.

13. Treatment 5:—A person, a man of 39 years old, used varenicline, commercial brand, at dose of 1 mg twice daily during 7 days, then 0.5 mg daily for the next 3 days; continuing with 0.5 mg twice a day during 4 days, and finally 1 mg during 1 day, every 12 h. This cycle was repeated 12 weeks. Treatment 5.

14. Anxiety was assessed by the Hamilton Anxiety Scale (Hamilton, 1969; Bulbena, 2000; APA, 2000) and the degree of nicotine dependence with Fagerstrom test (1991).

15. The Hamilton Anxiety Scale (Table 1) is one of the most used instruments in pharmacological studies of anxiety. It was used according to the criteria established by the American Psychological Association (APA, 2000), to assess the severity of anxiety in a global manner in patients who met criteria for anxiety or depression and to monitor response to treatment.

16. 14 scale items were evaluated, 13 related to anxiety symptoms and signs, and the last item values the patient's behavior during the assessment interview. In all cases it was applied by a medical doctor before and after each treatment (treatments 1 to 5, described in points 9 to 13 of this section). The interview lasted 25 to 30 minutes before and after treatment. Each item presents a number of signs and symptoms that are helpful in their assessment, although no specific anchor points. In each case both the item intensity and the item frequency were considered. Each item is rated on a scale of 0 to 4 points, since just some questions refer to signs that can only be observed during the interview, each patient was questioned about their situation in the last 2 weeks before the interview (both before starting treatment and once it was completed) for treatments 1 to 4, according to what reported by Bech (1993) and for the Treatment 5 (varenicline), lasting 12 weeks, the interview was applied 3 times, 1 time at the end of each month of treatment; always considering in each interview, the evaluation of the last two weeks before the new interview, according considerations Bech (1993).

17. The physician scored from 0 to 4 points each item, assessing both the intensity and the frequency of it. The total score was obtained from the sum of the scores of each of the items. The range is from 0 to 56 points. The scale does not provide cutoff points to distinguish people with and without anxiety, so the results were interpreted as a quantification of the intensity (Kellner, 1968; Lozano, 1990), resulting particularly useful variations after smoke quitting; the first evaluation in the case of 1 to 4 treatments, was performed on day 7 after smoke quitting; and in the case of the treatment 5, the evaluation was the fixed day for smoke quitting. The last assessment (2dn for treatments 1 to 4 and 3^{rd} for treatment 5) was performed after the treatment finish, a day after the last dosage, interviewing the patient about the signs and symptoms of the scale during the last two weeks, prior to the interview; the same period that lasted intake treatment in the case of 1 to 4.

18. According to Kellner (1968) two scores were obtained corresponding to psychological anxiety (items 1, 2, 3, 4, 5, 6 and 14) and the somatic anxiety (items 7, 8, 9, 10, 11, 12, and 13), since the effects of treatments can have different degrees of psychic and somatic symptoms (APA, 2000), resulting useful subscale scores, however for the purposes of this document, only the scale was considered for assessing generalized anxiety, asking about symptoms between anxiety attacks. So, under the criteria of Bech (1993) were considered, for guidance: 0 a 5 points “not anxious”, 6 to 14 “Anxiety minor” 15 or more “Anxiety greater.”.

19. Data were analyzed by statistical software, using ANOVA and statistically significant differences were obtained, then the results are shown:

20. The persons who used the treatment 1, ZAM® (activated and micronized zeolite) showed lower anxiety before and after treatment, even they stopped smoking; and reported less anxiety than they had the last time they tried to quit, both persons tried to quit smoking before with other kind of treatments. Tracking by phone and two weeks after completing the treatment 1, both persons remained without smoking.

21. The persons who used the Treatment 2, Raspberry Powder (registered patent application number Mx/a/2010/000827, file, folio MX/E/2010/004489) showed lower anxiety before treatment and not anxiety at the end of this. Both persons stopped smoking during the proposed period of treatment (21 days). Tracking was made by telephone every month, and even four months after the end of treatment, both persons still were able to be without smoking; one of them during the third month felt the desire of smoking, but was able to stay without smoking.

22. The person who used the treatment 3, a commercial product of raspberry, similar to the raspberry powder mentioned in the previous section in combination with ZAM® (activated and micronized zeolite) showed higher Anxiety before the treatment and minor Anxiety features at the end. The person quit smoking in the proposed period (21 days). Monitoring was conducted by telephone each month, and a month after the treatment, went back to smoke again; then this person asked to receive treatment again, which was provided to him and relapsed after a week, four months after the end of treatment, the person was still smoking.

23. In the case of the persons who used the treatment 4, raspberry powder (registered patent application number MX/a/2010/000827 file, folio MX/E/2010/004489) in combination with ZAM® (micronized and activated zeolite), one of them had higher anxiety before treatment and the other less anxiety. After treatment the first had lower anxiety and second No anxiety. Followed by telephone every month and four months after the end of treatment, both were still without smoking.

24. The person who used the treatment 5, varenicline showed less anxiety during the first month of treatment and increased anxiety during the second and third month. Monitoring by telephone every month was conducted, and the person reported that quit smoking in the second month of treatment, remaining smoke-free for two months; but also reported increased anxiety caused to fall smoking again, 1 month after treatment ends.

25. Fagerstrom test (Table 2) allows the measurement of the degree of physical dependence that smokers have for the nicotine, is one of the most significant findings in the clinical examination of smoking (Peto, 1996). Fagerstrom test has proven to be the most useful tool among those available to measure such dependence (Salleras, 1994). It has been the most universally used and with better quality parameters (Plans, 1995). This is a test with six items with multiple answers. Depending on the answer that each one of smoker gives to each of the questions you get a certain score. By sum the points earned in each of the issues a total score between 0 and 10 points is obtained.

TABLE 2

| Question | Answer | score |
|---|---|---|
| How much time passes | <5 minutes | 3 |
| between waking up and | 6-30 minutes | 2 |
| smoke your first cigarette? | 31-61 minutes | 1 |
| | After One hour | 0 |
| Find hard to stop smoking in places | Yes | 1 |
| where it is forbidden to do what? | No | 0 |
| Of all cigarettes consumed in the | The first in the morning | 1 |
| day, which needs more? | Any other | 0 |
| How many cigarettes | 31 or more | 3 |
| do you smoke a day? | 21-30 | 2 |
| | 11-20 | 1 |
| | 10 or less | 0 |
| After consuming the first | Yes | 1 |
| cigarette of the day, | No | 0 |
| smoke some more quickly? | | |
| Do you smoke as though this ill | Yes | 1 |
| need to stay in bed most of the day? | No | 0 |
| | TOTAL | |

| | |
|---|---|
| 0-1 | Very low physical dependence. |
| 2-3 | Low physical dependence. From this |
| | point it is convenient to use |
| | pharmacological treatment to |
| | achieve cessation of smoking. |
| 4-5 | Moderate physical dependence. |
| | A significant risk associated with |
| | consumption of snuff disease appears. |
| 6-7 | High physical dependence. |
| 8-10 | Extreme degree of physical dependence. |
| | Necessary to use drug therapy in |
| | parallel to a psychological treatment. |

26. The assessment of the test, not only serves to ascertain the degree of physical dependence that smoking has by the nicotine, but also can be used for prognostic purposes and therapeutic indication (Jiménez, 2000). The evaluation is done according to the following scheme: From 0 to 1 points the degree of physical dependence is very low. From 2 to 3 points shows a low degree of dependence. The use of treatment pharmacology for smoking cessation in this group of patients is helpful. Of 4-5 points indicates moderate degree of physical dependence on nicotine and a significant risk of illness associated with the consumption of tobacco. Approximately 30% of smokers have this score and in their attempts to quit tobacco consumption should use pharmacotherapy (Jiménez, 2000). The Smokers with 6 or 7 points suffer high physical dependence and are at high risk associated with the consumption of tobacco diseases. 15% of smokers get this score. According to Jiménez (2000) is essential to use drug therapy to quit smoking when making a serious attempt to abandonment. From 8-10 points indicate extreme degree of dependence. 5% of smokers get this score and your risk of developing associated diseases to tobacco consumption is very high.

27. In a research study (Prieto, 2003) concluded that 90% of smokers smoked daily and only 1, 7% did so weekly, finding that the average consumption was 15.21 cigarettes/day. 44% of smokers had nicotine dependence (assessed with a score equal to or greater than 5 on the Fagerström test), although the dependence was variable. The main motivation for continuing smoking was the pleasure (30.5%), followed by the routine habits (27.1%) and the feeling of relaxation (15.3%).

28. Under these considerations Fagerstrom test and treatment was given, as described above, to the 8 patients referenced herein. The following results were obtained:

29. Of those who used the treatment 1 (activated and micronized zeolite), one of them presented low physical dependence and other moderate physical dependence before treatment, both, as described before, quit smoking and after 14 days of treatment, both showed low physical dependence.

30. The persons who used the Treatment 2, (Raspberry Powder (registered patent application number Mx/a/2010/000827, folio MX/E/2010/004489) showed moderate physical dependence before treatment, and finished showing very low physical dependence. Tracking was made by telephone every month, and four months after the end of treatment, they still maintained without smoking.

31. The person who used the treatment 3 (a commercial product of raspberry, similar to the raspberry powder mentioned in the previous section in combination with ZAM® (activated and micronized zeolite) presented moderate physical dependence before treatment and low physical dependence at the end. However, tracking was made by telephone each month, and four months after finishing the treatment continue smoking.

32. Of the persons who used the treatment 4, (raspberry powder (registered patent application number Mx/a/2010/000827 file, folio MX/E/2010/004489) in combination with ZAM (micronized and activated zeolite), one moderate physical dependence before treatment and the other low physical dependence.

After treatment both showed very low physical dependence. Tracking was made by telephone every month, and four months after the end of treatment, they still were without smoking.

33. The person who used the treatment 5, varenicline presented moderate physical dependence before treatment, and high at the end of it. Tracking was made by telephone every month and reported having stopped smoking for 2 months, but then have fall smoking in the third month, and the person continue smoking.

34. The attention protocol of patients with treatments 1 to 4 was fundamental for best results, so that patent applicants, we are exposing to also protect this application. which is described below:

Patient care protocol.

35. On a first meet to patients is asked to them to list the reasons why you smoke and why you have to leave; on that same meeting, using a cognitive-behavioral process, the hazards and risks are explained that the snuff smoke implies for the health of himself and those around him; He need to set a date to quit smoking and is given a list of circumstances that allow you to measure the chances of success and they are required to analyze them and fill out the questionnaire containing this list before going to the next appointment.

36. In a second date is discussed with the patient the date to quitting and is reinforced on the fact that should be a significant date for him, while analyzes and reinforces the list of circumstances that speak of the possibilities successful smoking cessation. Selected the date is programmed a third date, it will happen 5 days before the date fixed for smoking cessation program.

A list of activities to be solved by the patient before going to the third date come is given, prompts make a diary in which you must record each cigarette smoked and why it does; are asked to think, before lighting each cigarette, because it does; is asked to whether it is really necessary, and if you considered this you can smoke. You are asked to delay a little every day the first cigarette of the morning. You are instructed not to accumulate snuff, advising buy one package at a time. You are invited to try smoking in a spaced form and not smoke the cigarettes that would not be entirely necessary and to turn them off in the middle. You are instructed to refuse offers of snuff other smokers and think of the fact that although fall into any of the deals, gradually get used to reject them. It makes you reflect on snuff trap “light”, indicating that the fact of having less nicotine can lead to smoke more often and take deeper puffs. You are instructed to practice any physical activity you can do regularly (walking, running, cycling, etc.). You are invited to comment to the people around you that you will quit in a few days. You are encouraged to seek company and to form a group to know that you can support to overcome the bad times together. It makes you think you can ask family and/or friends who are part of the support group, who can go, even by telephone, to overcome some point of crisis, the physician and/or therapist phone are provides the or treating institution.

37. From that moment appointment is given 5 days prior to the date fixed for quitting. On the third appointment, is forewarned that from this moment you have 5 days to quit and reviewed with the patient what they should do every day:

38. First Day, should set the time to quit. You are invited to talk again with your friends and family of your plan. You are prompted to stop buying cigarettes and write the date in a “Letter of Commitment”, which specify the time, day, month and year will be to quit smoking.

39. Second Day You are prompted to make another list of when and why you smoke. It invites you to think of that day in new ways to relax, things can get his hands instead of a cigarette, you are invited to analyze habits or routines that you want to change and are requested write this into a List.

40. Third Day. will be show on that day make a list of things you can do with the money you'll save by quitting. You are invited to call a friend, an ex-smoker or your support group when you need help.

41. Fourth Day. You are told to take 1 capsule of 500 mg of composed Valerian each/8 hours. For 5 days. You are instructed to wash their clothing, and bedding to remove cigarette smell.

42. Fifth Day is suggested that you choose a reward and the acquiring, to give it to yourself after you quit. You are instructed to make an appointment with your doctor and/or treating institution. It tells you that the end of the day all cigarettes and matches be discarded, and keep lighters and ashtrays

43. Final Day. This day is the date set in the first day. It tells you kept well occupied, you change your routine when possible and do things out of the usual. It tells you to remind your family, friends and work colleagues that this is the day to quit and invites you to ask them to give you help and support.

44. Day After. Congratulate yourself and your reward be purchased, a gift or do something to celebrate. You are instructed to avoid alcohol. It is suggested that when you want a cigarette, you do something that is not related to smoking, such as a walk in the park, take a glass of water, or deep breathing.

45. Upon completion of the third appointment are delivered in writing the above indications and gather to the day after the date for quitting. Are also given, in writing, information on what to do in case of anxiety, constipation, hunger and/or weakness.

46. The fourth appointment is scheduled for the 6th day, after starting the countdown 5 days to the date specified has quit. At this appointment the patient is advised about treatments that support the process of quitting, advantages and disadvantages are communicated. Are explain what the process of detoxification of the body and is guided on using the treatment with the product, resulting from the combination of the ZAM® (micronized and activated zeolite), as described in the trademark registration, and raspberry powder (according to process registered in the Mexican Institute of Industrial Property, in patent application number Mx/a/2010/000827 record dated 21/ENE/2010, folio MX/E/2010/004,489), subject to registration of this patent document. Treatment with this product is indicated for 14 days at doses of 7 g of powder diluted, preferably in fruit juice or any other drink except alcohol and suggesting that ingested daily at the same time, preferably in the morning.

47. The base powder reconstituted with water retains its content of minerals such as calcium, magnesium, potassium, silicon, sodium, manganese, sulfates and phosphates, carbohydrates and nitrogenous compounds, as well as vitamins A, B1, B2 and C, the latter will give the quality of being an excellent antioxidant to counter free radicals, being vitamins preferred, but it is feasible and it is permissible to add any other vitamin supplement. Likewise retains chemical components ZAM® (activated and micronized zeolite) SiO2, CaO, K2O, MgO, Na2O by strengthening antioxidant and detoxificant effect by be mineral negatively charged microcrystalline and naturally attract and adsorb positively charged toxicants because either air or the skin and blood. The product covered by this application provoke benefic effects in health in the process of abandon of smoking interfering in the anxiety caused by nicotine withdrawal, and need of nicotine and the need to keep using nicotine as stimulant and in the detoxify the body of this substance and others present in the tobacco smoke. It can be used diluted in water or in any permissible drink except alcohol, or as an additive in any food such as cookies, jellies, cakes, gelatins, yogurt, among others, as well as other personal care products such as oral rinses, toothpaste, and skin creams, among others cleaning products like laundry detergents, soap for the whole body, shampoo, among others, cigarette filters and others.

48. The base powder has a shelf life of more than one year while retaining its original properties.

Having sufficiently described the invention, it is considered as a new product, process and therapeutic properties obtained from it and therefore claim as our exclusive property contained in the following claims.

1-35. (canceled)

36. A method for producing a raspberry base chemical compound for use in treatment of anxiety symptoms, the method comprising:
selecting a raspberry;
preparing the raspberry;
obtaining a raspberry extract from the raspberry;
adding a thickener agent to the raspberry extract
adding a zeolite to the thickener agent and the raspberry extract to form a fluid mixture; and
dehydrating the fluid mixture to form a powder.

37. The method of claim 1, wherein the step of obtaining the raspberry extract is achieved through a method selected from the group consisting of:
a marmite method, whereby a crushed raspberry fruit compound is slowly heated to a temperature not exceeding 80° C.; or
a bath method; whereby a crushed raspberry fruit compound is placed in a water bath, whereby a temperature of the crushed raspberry fruit compound does not exceed 80° C.

38. The method of claim 1, comprising the additional step of adding a sweetener to fluid mixture prior to dehydrating.

39. The method of claim 1, wherein the dehydrating of the fluid mixture is through a spray drying process using a spray-dryer equipment to an inlet temperature of 110° C. and outlet of 80° C., with an air flow of 600 ml/min and vacuum 30 millibars.

40. The method of claim 1, wherein the added zeolite is a clinoptilolite zoelite.

41. The method of claim 38, wherein the sweetener is selected from the group consisting of:
sugar;
fructose;
aspartame; or
combination thereof.

42. The method of claim 38, comprising the additional step of adding an anti-humactant to the fluid mixture prior to dehydrating.

43. The method of claim 1, wherein the zeolite is thermo-activated micronized zeolite-clinoptiolita with an average size of 3 micras and a superficial size of 24 m²/g.

44. A raspberry base chemical compound for use in treatment of anxiety symptons produced by a method comprising the steps of:
selecting a raspberry;
preparing the raspberry;
obtaining a raspberry extract from the raspberry;
adding a thickener agent to the raspberry extract
adding a zeolite to the thickener agent and the raspberry extract to form a mixture; and
dehydrating the mixture to form a powder.

45. The method of claim 44, the mixture being a fluid.

46. The method of claim 44, the mixture being a powder.

47. The compound of claim 43, wherein the step of obtaining the raspberry extract is achieved through a method selected from the group consisting of:
a marmite method, whereby a crushed raspberry fruit compound is slowly heated to a temperature not exceeding 80° C.; or
a bath method; whereby a crushed raspberry fruit compound is placed in a water bath, whereby a temperature of the crushed raspberry fruit compound does not exceed 80° C.

48. The compound of claim 44, comprising the additional step of adding a sweetener to fluid mixture prior to dehydrating.

49. The compound of claim 48, wherein the sweetener is selected from the group consisting of:
sugar;
fructose;
aspartame; or
combination thereof.

50. The compound of claim 44, comprising the additional step of adding an anti-humactant to the fluid mixture prior to dehydrating.

51. The compound of claim 44, configured to be rehydratable in liquids.

52. A raspberry base chemical compound for use in treatment of anxiety symptoms comprising:
raspberry extract;
thickener agent; and
zeolite.
